(19) United States
(12) Patent Application Publication
(10) Pub. No.: US 2021/0030299 A1
(43) Pub. Date: Feb. 4, 2021

(54) APPARATUS AND METHODS FOR DETECTION OF THE ONSET AND MONITORING THE PROGRESSION OF CEREBRAL ISCHEMIA TO ENABLE OPTIMAL STROKE TREATMENT

(71) Applicant: THE BOARD OF TRUSTEES OF THE LELAND STANFORD JUNIOR UNIVERSITY, Stanford, CA (US)

(72) Inventors: Urs Hendrik NABER, Redwood City, CA (US); Orestis VARDOULIS, Palo Alto, CA (US); Patrick Clyne THOMPSON, San Francisco, CA (US); Lily Ayodele ROBERTS, Palo Alto, CA (US)

(21) Appl. No.: 17/045,560

(22) PCT Filed: Apr. 8, 2019
(86) PCT No.: PCT/US2019/026367

§ 371 Date: Oct. 6, 2020

Related U.S. Application Data
(60) Provisional application No. 62/653,829, filed on Apr. 6, 2018.

(51) Int. Cl. A61B 5/0478; A61B 5/00

(52) U.S. Cl. CPC A61B 5/0478; A61B 5/7267; A61B 5/6868; A61B 2562/0247; A61B 5/7203; A61B 2562/0219; A61B 5/7405

(57) ABSTRACT

Provided herein are systems and methods for automatically determining if a subject is having a stroke. A subject's brain may be monitored with sensors or electrodes to acquire biopotential voltage data. EEG data, power spectrum data and ratios and differences thereof, principal component analysis (PCA) features, and/or other engineered features may be extracted scored with automated machine learning systems. A classifier can identify and/or output if the subject is having a stroke. The subject or a third party can be automatically notified that the subject is having a stroke.

### CROSS REFERENCE TO RELATED APPLICATIONS

This application claims the priority of U.S. Provisional Patent Application No. 62/653,829, titled “APPARATUS AND METHODS FOR DETECTION OF THE ONSET AND MONITORING THE PROGRESSION OF CEREBRAL ISCHEMIA TO ENABLE OPTIMAL STROKE TREATMENT,” filed Apr. 6, 2018, which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

A cerebrovascular accident (CVA) or stroke refers to the loss of brain function secondary to inadequate oxygen supply, most commonly due to disruption in perfusion to the brain. With lack of oxygenation, there is subsequent energy depletion, resulting in loss of cellular membrane potential and subsequent depolarization of neurons and glial cells. Within minutes, there is irreversible damage to the ischemic tissue core which is supplied by inline flow. The surrounding unstable tissue region, termed the penumbra, is rendered dysfunctional by restricted blood supply and oxygen, but retains marginal energy metabolism due to collateral blood flow. This tissue is potentially salvageable if proper blood flow can be restored, but the collateral blood flow is unstable and with time will be inadequate for neuronal survival. It is well understood that time to treatment inversely correlates with better outcomes. With each second of continued ischemia, the core grows, including more of the penumbra until there is no salvageable tissue left. The penumbra is the target of current revascularization therapy, as this at risk tissue can recover function given restoration of oxygen and energy supply.

Stroke can be categorized as ischemic or hemorrhagic. Ischemic stroke, which constitutes roughly 87% of all strokes, is mainly secondary to atherothrombotic, embolic, and small-vessel diseases. Less common causes include coagulopathies, vasculitis, dissection, hypotension and venous thrombosis. Hemorrhagic stroke occurs secondary to vessel rupture leading to either an intracerebral or subarachnoid hemorrhage, with direct damage of the surrounding tissue as well as impairment of blood flow to the tissues distal to the hemorrhage that may cause cerebral ischemia.

Stroke is the fifth leading cause of death in the United States and second leading cause of death worldwide. In the United States, there are roughly 795,000 strokes annually and 7 million stroke survivors, a number expected to grow rapidly as the population ages. Stroke survivors suffer from substantial morbidity. Stroke is the leading cause of long-term disability in the United States accounting for roughly $72 Billion in direct medical care today. The AHA estimates the annual direct medical cost of stroke to increase to as much as $180 Billion by 2030.

As outlined above, functional outcomes, survival, and cost of care all improve the earlier blood flow can be restored to the tissue at risk. “Time is brain” is a widely adopted maxim, and door to needle times have become a quality marker for stroke centers around the world. Delays can happen twice, before arrival at the stroke center. First, the patient or someone around the patient needs to identify the stroke and activate help. Second, the patient needs to be transported to the treatment facility. Unfortunately, despite public awareness campaigns, the majority of delays happen due to inadequate recognition or other circumstances in the patient's home. A large percentage of patients present to the hospital too late for effective treatment.

### SUMMARY OF THE DISCLOSURE

Implementations address the need to provide an automated stroke detection system to automatically, effectively, and accurately assess the onset and/or progression of cerebral ischemia, with a high degree of accuracy. The present application addresses these and other technical problems by providing technical solutions and/or automated agents that automatically identify stroke in patients to improve the accuracy and rapidity of stroke identification. Automatic stroke detection may further enable automatic alerts and/or notifications to the patient, caregivers, loved ones, or emergency and/or medical personnel when a stroke has been detected.

In general, example apparatuses (e.g., devices, systems, etc.) and/or methods described herein may acquire EEG data from a patient's brain for use as the raw features in a scoring model. The raw features may be, for example, continuous EEG recordings from the patient's brain. The EEG data can be acquired with devices or systems that include electrodes implanted or placed on or proximate to the patient's brain. For example, electrodes can capture EEG data while positioned proximate to, one, two, three, or four principal lobes on each side of the patient's brain, including the frontal, temporal, occipital and parietal lobes.

Furthermore, example apparatuses (e.g., devices, systems, etc.) and/or methods described herein may further apply pre-processing to the raw features for the scoring model. For example, automated agents may reduce or eliminate noise and other irregularities from the EEG data. The preprocessing can provide a modified set of raw features to the scoring model for automatic stroke detection.

In general, example apparatuses (e.g., devices, systems, etc.) and/or methods described herein may implement feature engineering with the raw features to build the scoring model that provides automatic stroke detection. There are many approaches to do feature engineering for building a scoring model, including hand designed features of Power Spectral Density (PSD) and ratios and spatial/temporal differences thereof, and hand designed brain connectivity metrics and spatial/temporal differences thereof, and hand designed features of Principle Component Analysis (PCA) and spatial/temporal differences thereof, and automated feature exploration (e.g., using deep neural networks or other feature selection methods) and spatial/temporal differences thereof, and a hybrid feature engineering approach, namely combining hand designed features and automated feature exploration model architecture and spatial/temporal differences thereof.

In general, example apparatuses (e.g., devices, systems, etc.) and/or methods described herein may use machine learning classification models with the engineered feature data to return a score on whether a stroke is occurring. Examples of machine learning systems that may be used include, but are not limited to, Convolutional Neural Networks (CNN), Decision Tree, Random Forest, Logistic Regression, Support Vector Machine, AdaBoosT, K-Nearest Neighbor (KNN), Quadratic Discriminant Analysis, Neural Network, etc. The machine learning classification models can be configured to apply ground truth labeling when generating an output data set or stroke detection score. In some examples, the machine learning classification model can output a binary scoring label (e.g., output of 1 for a stroke and output of 0 for not a stroke). In another example, the machine learning classification model can output a linear scale rating (e.g., the likelihood of a stroke between 0 and 10). In yet another example, the machine learning classification model can output a multilabel classification score (e.g., “stroke”, “no stroke”, as well as additional classification such as “non-stroke/seizure”, “non-stroke/walking”, “non-stroke/sleep”, “non-stroke/chewing”, “non-stroke/blinking”, “non-stroke/facial muscle activity, etc.).

A “patient,” as used herein, may be any subject (e.g., human, non-human, adult, child, etc.) and may be alternatively and equivalently referred to herein as a “patient” or a “subject.” A “patient,” as used herein, may but need not be a medical patient. A “patient,” as used herein, may include a person who is at risk of having a cerebral ischemic event, stroke, or onset of stroke.

The terms “automatically” and “automated” as used herein, may be any process, technique, method, algorithm, or set of instructions that is performed or implemented by a computer system or is computer-aided. In embodiments herein, the implementation of automatic and automated processes, methods and the like, can provide improved detection, identification and response times to an extent that would not be achieved solely with manual (human-only) processes.

As will be described in greater detail herein, automatically detecting the onset of stroke may include collecting EEG data from the patient's brain. Collecting the EEG data may include implanting a stroke detection device on or in the patient, including implanting electrodes on or proximate to the patient's brain. Collecting the EEG data may further include receiving, measuring, detecting, and/or recording EEG data directly (e.g., using electrodes) or indirectly (e.g., acquiring the EEG data from a separate device and/or third party, acquiring the EEG data from a memory, or the like).

Additional information may be collected or input into the devices and systems described herein, including patient information (e.g., age, gender, risk factors for stroke, contact information for the person and/or the caregivers/loved ones etc.).

The EEG data may be processed to extract one or more features that may be used to automatically detect the onset of stroke in the patient.

Any appropriate features may be extracted from the EEG data. For example, in some variations, features may include a principal component analysis (PCA) of the EEG data. Additional features may not be necessary (e.g., PCA alone may be used) or it may be used to supplement the PCA of the EEG data. PCA may be performed on the EEG data automatically using any appropriate technique, as discussed above, including using modules from existing software environments such C++ and C# (e.g., ALGLIB library that implements PCA and truncated PCA, MLPACK), Java (e.g., KNIME, Weka, etc.), Mathematica, MATLAB (e.g., MATLAB Statistics Toolbox, etc.), python (e.g., numpy, Scikit-learn, etc.), GNU Octave, etc.

Note that although there are examples provided herein using PCA, other eigenvector-based multivariate analyses may be used. PCA may be automatically performed using known techniques including computing PCA using a correlation technique and/or a covariance technique, iterative methods including but not limited to non-linear iterative partial least squares techniques.

A method of automatically determining if a subject is having a stroke is provided, the method comprising the steps of acquiring, in a computing device, biopotential voltage data from the subject's brain, determining, in the computing device, [EEG features or metrics from the biopotential voltage data, creating, in the computing device, engineered features from the EEG features or metrics, applying the engineered features to a classifier of the computing device, outputting from the computing device a stroke score for the subject, and in the event that the stroke score indicates that the subject is having a stroke, providing feedback that the subject is having a stroke.

In some examples, the feedback comprises sending a notification to the subject or a third party that the subject is having a stroke. In one embodiment, wherein the notification comprises a digital text message to an external device that the subject is having a stroke. The notification can further include relevant information about the subject selected from the group consisting of age, gender, physical description of the subject, and physical location of the subject. In another example, the notification comprises a voice or audible message to an external device that the subject is having a stroke.

In one example, outputting comprises outputting a binary score indicating that the subject is likely having a stroke or likely not having a stroke. In another example, outputting comprises outputting a linear score indicating along a scale how likely it is that the subject is having a stroke.

Creating engineered features may further comprise taking a principal component analysis (PCA) of the EEG features or metrics. In other examples, creating engineered features comprises power spectral density (PSD) ratios and channel connectivity metrics and spatial/temporal differences thereof.

In one embodiment, the biopotential voltage is acquired from at least one electrode implanted under the scalp of the subject.

In some examples, applying the engineered features to the classifier comprises applying either a binary classifier or a linear classifier to the engineered features.

A non-transitory computing device readable medium having instructions stored thereon for determining if a subject is having a stroke is also provided, wherein the instructions are executable by a processor to cause a computing device to acquire biopotential voltage data from the subject's brain, determine EEG features or metrics from the biopotential voltage data, create engineered features from the EEG features or metrics, apply the engineered features to a classifier of the computing device, output a stroke score for the subject, and in the event that the stroke score indicates that the subject is having a stroke, provide feedback that the subject is having a stroke.

In one embodiment, the feedback comprises a notification to the subject or a third party that the subject is having a stroke. In some examples, the notification comprises a digital text message to an external device that the subject is having a stroke. In another example, the notification includes relevant information about the subject selected from the group consisting of age, gender, physical description of the subject, and physical location of the subject. In some embodiments, the notification comprises a voice or audible message to an external device that the subject is having a stroke.

In another embodiment the instructions are further configured so that the output is a binary score indicating that the subject is likely having a stroke or likely not having a stroke.

In some examples, the instructions are further configured so that the output is a linear score indicating along a scale how likely it is that the subject is having a stroke.

In another example, the instructions are further configured to create engineered features by taking a principal component analysis (PCA) of the EEG features or metrics.

In other examples, the instructions are further configured to create engineered features with power spectral density (PSD) ratios and channel connectivity metrics and spatial/temporal differences thereof.

In one embodiment, the biopotential voltage is acquired from at least one electrode implanted under the scalp of the subject.

A device configured to detect the onset of stroke is also provided, comprising at least one electrode configured to be implanted under a subject's scalp to measure biopotential voltage of the subject's brain, a control unit electrically coupled to the at least one electrode, the control unit comprising a processor, a power source, communications hardware configured to transmit and receive data to and from an external device, and at least one memory storage device disposed in a housing, wherein the control unit is configured to acquire, in the at least one memory storage device, biopotential voltage data from the at least one electrode, determine, with the processor, EEG features or metrics from the biopotential voltage data, create, with the processor, engineered features from the EEG features or metrics, apply, with the processor, the engineered features to a classifier stored on at least one memory storage device, output, with the processor, a stroke score for the subject, and in the event that the stroke score indicates that the subject is having a stroke, provide, with the communications hardware, feedback that the subject is having a stroke.

In one embodiment, the feedback comprises a notification to the subject or a third party that the subject is having a stroke.

In some embodiments, the power source is a battery. In specific embodiments, the battery is selected from the group consisting of a long lasting battery, a replaceable battery, and a rechargeable battery. In some embodiments, the battery is removable from the control unit. In other embodiments, the battery is rechargeable by induction or by ultrasound.

In some examples, the at least one memory storage device comprises short term and long term data storage.

In some examples, the external device is selected from the group consisting of a smartphone, a tablet, a computer, or a server.

In one example, the device further comprises at least one sensor.

In some embodiments, the sensor is an accelerometer, a pressure sensor, a microphone, or a GPS sensor.

In some examples, the at least one electrode comprises at least 2 electrodes configured for placement on each temporal lobe on the subject's brain. In another example, the at least one electrode comprises at least 2 electrodes configured for placement on each frontal parietal lobe of the subject's brain.

In one embodiment, the at least one electrode has a thickness between about 10 microns and about 2 mm.

In other examples, the at least one electrode comprises a flexible conductive material suitable for conforming to the surface of the subject's skull or brain. In one embodiment, the at least one electrode comprises a metal selected from the group consisting of gold, platinum, and silver oxide.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is one example of a stroke detection system.

FIG. 1B is a schematic diagram showing hardware components of a control unit of a stroke detection system.

FIGS. 1C-1D illustrate one example of sensor assemblies joining a control unit of a stroke detection system at a connector.

FIGS. 1E-1G show various views of sensor assemblies of a stroke detection system, including electrodes, leads, and a substrate.

FIG. 2A is a diagram showing an example of a computing environment configured to determine if a patient is having a stroke.

FIG. 2B is a diagram showing an example of preprocessing engine(s).

FIG. 2C is a diagram showing an example of a feature extraction engine(s).

FIG. 2D is a diagram showing an example of a scoring engine(s).

FIG. 2E is a diagram showing an example of a notification engine(s).

FIG. 3 is a flowchart describing one example of determining if a patient is having a stroke.

FIG. 4 is another flowchart describing one example of determining a stroke score.

### DETAILED DESCRIPTION

Described herein are apparatuses (e.g., systems, computing device readable media, devices, etc.) and methods for detecting, identifying, and notifying a patient or others about the onset of stroke in the patient. One object of the present disclosure is to use machine learning technology to provide an automatic classifier that can assess the onset of stroke. The classifier can make this determination based upon data including EEG data, patient demographics, processed EEG features, and historical patient data. These methods and apparatus can use this information to provide a stroke detection feedback to a patient, physician, loved one, family member, emergency services (e.g., police, fire, paramedic, etc.), or the like.

For example, described herein are apparatuses and/or methods, e.g., systems, including systems to automatically implement processes that incorporate a stroke detection system. The stroke detection system can measure, record, and/or retrieve EEG data from a local or remote database, process the EEG data, and convert the EEG data into engineered features. The features can then be passed into a stroke detection classification model, which may use machine learning technology (e.g., Convolutional Neural Network (CNN), Decision Tree, Random Forest, Logistic Regression, Support Vector Machine, AdaBOOST, K-Nearest Neighbor (KNN), Quadratic Discriminant Analysis, Neural Network, etc.) to return a score on whether a stroke is occurring or monitoring has to be intensified, to improve predictive power. The parameters inputted into the stroke detection classification model can be optimized with historic data. The stroke detection classification model may be used to provide an output indicating that a stroke is occurring. The results may be provided on demand and/or may be stored in a memory (e.g., database) for later use.

The apparatuses and/or methods (e.g., systems, devices, etc.) described below can be configured to automatically deliver feedback or a notification alert when a stroke has been detected. Automatic feedback, alerts, and/or notifications may be sent or delivered to the patient, caregivers, loved ones, or emergency and/or medical personnel. In some embodiments, the stroke detection system includes wireless communication systems that enable direct notifications to the patient or others. For example, the wireless communication systems can include Bluetooth, Wi-Fi, cellular, Low Power Wide Area Network (e.g. NB-IOT, LoRa), or other wireless communication protocols and hardware. In some examples, the notification can include a prompt or notice to the patient or a loved one/caregiver that the patient is having a stroke and to call emergency services on behalf of the patient. In other examples, the stroke detection system can directly notify emergency services that the patient is having a stroke, this can happen either through direct link to EMS or an intermediary, such as a stroke response center. The notification can further include information pertaining to the patient, including a precise location, name, age, description, etc. The notification can be customized to ensure that the person/entity being notified can quickly and accurately locate the patient for transportation to a hospital or medical care facility. The notification can be customized to include information on the location, severity and growth rate of the neurologic injury. The notification can be further customized to include information about the appropriate level of medical care based on the characteristics of the captured stroke event. In some situations, the patient may have opted out of transportation by ambulance. In this case, the notification can be configured to share information with a ride-sharing or transportation service to hail or request transportation from the patient's location to a hospital or medical treatment facility.

As described herein, a stroke detection system can measure, record, process, and store continuous EEG data from a patient. The stroke detection system can include hardware that is implanted on or within a patient to measure EEG data. The hardware can include, for example, one or more electrodes implanted under the scalp of the patient, and a control unit with computer hardware, software, and/or firmware configured to process, record, and store the EEG data. The control unit can also be implanted under the scalp of the patient, or alternatively, can be located partially or entirely external to the patient's body.

A stroke detection scoring system, as used herein, may include a system that uses automated agents to identify the onset or occurrence of stroke in a patient from EEG data. The system can include self-improving algorithms or be improved through periodic firmware upgrades, either wirelessly or through direct link. The present disclosure presents one or more novel processes for identifying stroke. Some implementations herein may solve technical problems related to optimizing and/or increasing the accuracy of stroke detection.

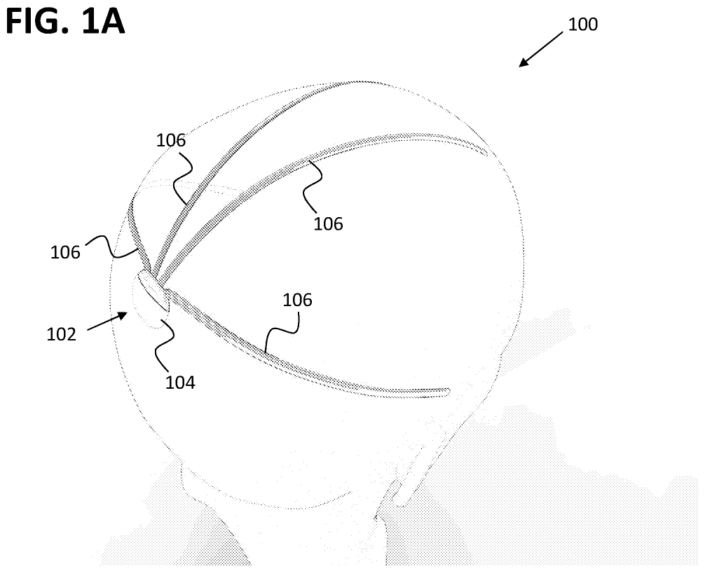

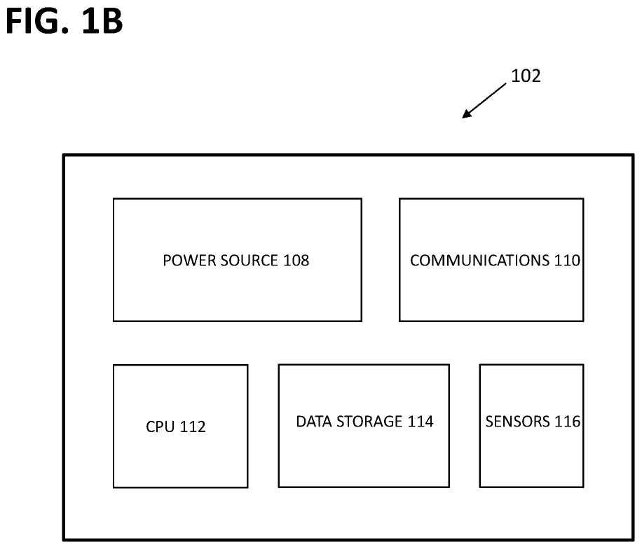

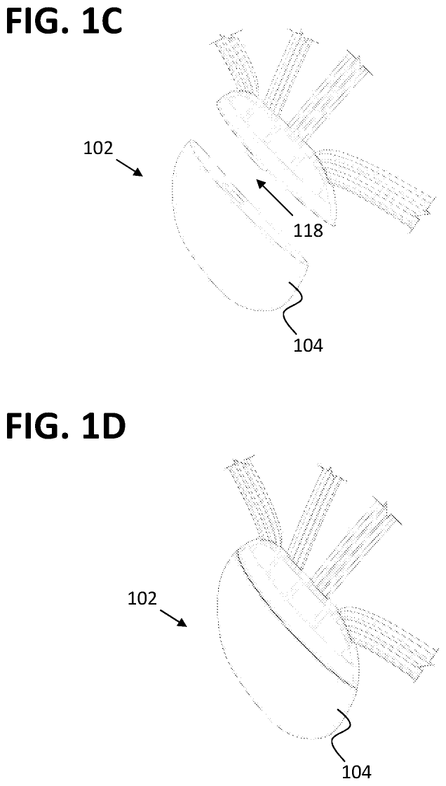

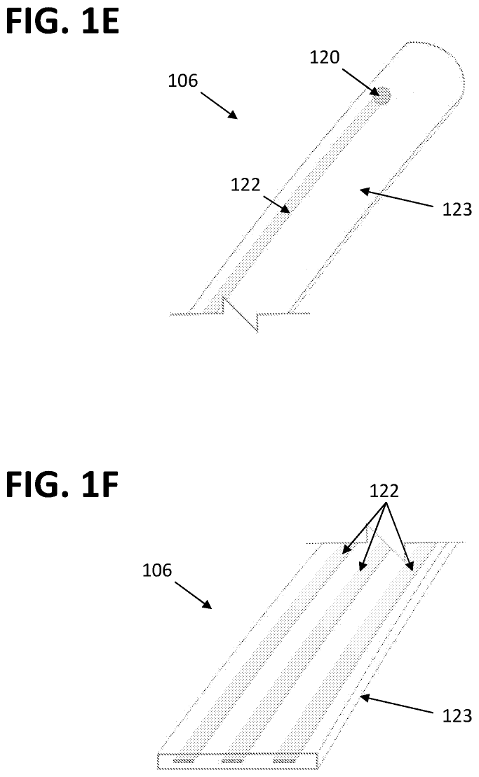

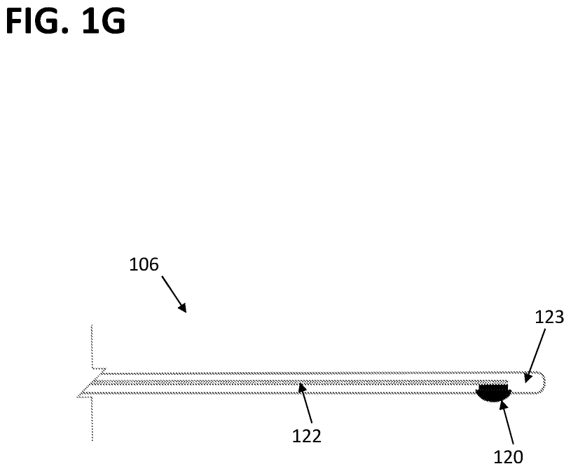

FIG. 1A is a drawing showing one example of a stroke detection system 100, which can include a control unit 102 disposed within housing 104 and one or more sensor assemblies 106 electrically and physically coupled to the control unit. The sensor assemblies can each comprise, for example, at least one electrode and an electrical lead connecting the electrode to the control unit. Four sensor assemblies are shown in the example of FIG. 1A, providing at least one electrode per lobe of the brain. In this example, two sensor assemblies are positioned on the side of the head, to extract signals from the temporal and occipital lobes of the brain, and two sensor assemblies are positioned towards the top of the head, extract signals from the frontal and parietal lobes. In some embodiments, more or fewer sensor assemblies can be implemented. For example, bilateral sensor arrays, semicircular sensor arrays, and multi-sensor arrays can also be used. The stroke detection system can include, for example, 1, 2, 3, 4, 5, 6, 7, 8, 9, 10, 11-16, or 17-32 sensor assemblies.

In the illustrated embodiment, the entire stroke detection system 100, including the control unit 102, housing 104, and sensor assemblies 106 are implanted under the skin of the patient, specifically in the loose areolar tissue of the scalp. In other embodiments, some or all components of the stroke detection system can be located or positioned external to the patient. For example, the sensor assemblies may be disposed under the skin of the patient, while the control unit may be positioned outside of the patient. Alternatively, the control unit and sensor assemblies could be disposed on, not under, the skin. The stroke detection system can also be implanted or located at other parts of the body, including in the sinuses, the oral cavity, behind the ear, or in the middle ear.

The control unit 102 can include electronics and computer hardware, as well as software/firmware configured to facilitate receiving, processing, storing, and outputting EEG data from the sensor assemblies 106. As shown in FIG. 1A, in an exemplary embodiment the control unit 102 is located at a posterior portion of the patient's head under the scalp in the loose areolar tissue of the subgaleal space. The control unit can include a housing that is designed with a thickness and size to be relatively imperceptible to the patient when implanted under the scalp. For example, in one example, the control unit housing can have dimensions in the range of 1-10 mm height by 20-35 mm length by 5-15 mm width. Patient comfort can be improved depending on the location of the implant. For example, the implanted location of the control unit in FIG. 1A is at the flattened posterior portion of the skull, above the lambdoid suture line, proximal to the posterior fontanel. This position can be beneficial to prevent pressure on the control unit or tissues when pressure is applied to the back of the head, such as when the patient is lying supine.

FIG. 1B is a schematic diagram showing the hardware components of control unit 102, including power source 108, communications 110, CPU 112, memory 114, and sensor(s) 116.

The power source 108 can be a battery or other power source, providing power to the control unit. In some embodiments, the power source can be configured to provide power to the control unit for a prolonged period of time (e.g., 4-6 years) before requiring replacement. In other embodiments, the power source is rechargeable, either by direct electrical connection or by wireless charging. In other embodiments the power source is replaceable, for example through a minor surgery that involves accessing the implanted control unit and replacing the battery.

Communications 110 can include processors, antennas, and/or radios configured to transmit and/or receive wireless information. In some examples, communications 110 can include Wi-Fi, Bluetooth, near field communication (NFC), and/or cellular communications hardware. For example, the communications 110 can be configured to wirelessly transmit a notification or alert to an external device when the stroke detection system detects the onset of a stroke. In one example, the communications 110 can use cellular hardware to directly alert a loved one or caregiver of the patient, the patient, or emergency services that the patient is having a stroke. The alert or notification can be, for example, a sms/mms message, a telephone call with a predetermined or prerecorded message, or the like. The alert or notification can include relevant information to assist in providing emergency care to the patient, including the patient's name, location, age, physical description, etc. The alert or notification can include information on the severity of the event along with the appropriate level of hospital care. In other embodiments, the alert or notification can be sent to a remote device via Bluetooth or wireless, such as a tablet, smartphone, computer wearable, or personal computer (pc).

CPU(s) 112 can be one or more computer processing units configured to carry out the instructions of the stroke detection system, including receiving EEG data from the electrodes, processing the EEG data, applying the processed EEG data to a classifier, determining if the patient is experiencing a stroke, and notifying a person or persons that the patient is having a stroke. The CPU(s) 112 can be electrically coupled to the other components of the control unit to control the functions of those components. For example, the CPU can manage power from the power source, control communications with communications 110, control storage of data in memory 114, and receive and process information from sensors 116. The CPU(s) 112 can enable hardware implementation of signal processing directly on the control unit of the stroke detection system. In some examples, FPGAs can be used which can be re-programmed remotely as the techniques and methods of the stroke detection system and algorithms are refined.

Memory 114 can comprise physical computer hardware configured to store software and instructions necessary for the operation of the control unit (e.g., operating system), data produced and processed by the control unit itself (e.g., EEG data from the electrodes), patient information (e.g., age, physical description, gender, etc.), and historical patient data. Memory 114 can include both volatile memory and non-volatile memory. In some examples, memory 114 can be configured to store a large amount of continuous EEG data for the patient. When the memory allocated to storing EEG data runs out, the memory can be configured to overwrite old EEG data. In other embodiments, the control unit can be configured to periodically connect to remote cloud storage for storage of all continuous EEG data. This can be implemented, for example, by periodically connecting the control unit to Wi-Fi and uploading all the EEG data stored in memory 114, or while being in proximity to a wireless/inductive charging system by Bluetooth, NFC, Wi-Fi, or cellular connectivity or via a Low Power Wide Area Network protocol (e.g. NB-IOT or LoRa).

Sensor(s) 116 can be various types of sensors that can aid in automated stroke detection or improve the performance or usability of an implanted control unit. For example, the control unit can include one or more accelerometers to provide a metric about the patient's movement before, during, or after a stroke event. As one example, an accelerometer could provide information that a patient has fallen and is not moving after the system detects a stroke event. This information could be passed on to the appropriate person (e.g., caregiver, loved one, emergency services) as an additional data point on the severity of the stroke event. The accelerometer could also be used as an input for an adaptive filtering method (e.g., Kalman Filter) to reduce and/or remove motion induced artifacts and signal noise from the acquired EEG data. Pressure sensors can also be included in the control unit, for example, to provide a warning to the patient or another if too much pressure is applied to the control unit (e.g., lying directly on the control unit during sleep, etc.) or to inform the stroke identification algorithm that some of the electrodes might be experiencing excessive noise due to pressure induced artifacts. In one implementation a thermal sensor on the control unit (102) can limit processing power and control heat generation to protect surrounding tissue. In one implementation sensors may include one or more microphones to deduct pulsation of the brain vessels or speech generation by the patient. In one implementation, the sensor(s) 116 can include GPS sensors to determine an accurate geo location of the patient. This GPS information can be transmitted along with the stroke alert/notification to a third party to indicate the precise location of the patient having a stroke. Geo location can be improved by triangulation via the cellular communication network. In another implementation triangulation via the cellular communication network can be used without GPS to infer the location of the patient having a stroke and the information can be transmitted along with the stroke alert notification to a third party.

In some examples, the control unit is permanently attached and/or connected to the sensor assemblies. In this scenario, the entire stroke detection system must be implanted at once, and if a component fails or malfunctions then the entire system must be removed and replaced. In another embodiment, shown in FIGS. 1C-1D, the sensor assemblies can join at a connector that removably attaches to the housing 104 of the control unit 102, as shown. The connector can provide electrical energy from the control unit to the sensor assemblies and can also transmit data measured by the sensor assemblies to the control unit. FIG. 1C shows the control unit separate from the connector 118 and sensor assemblies, and FIG. 1D shows the connector 118 physically attached to the control unit 102. In this embodiment, if a single component of the stroke detection system fails or requires replacement (e.g., the control unit, battery, etc.), then the failed component can be replaced without having to remove the entire system from the patient. It is particularly advantageous to not have to replace the sensor assemblies in the event of a failure in the control unit, since implantation of the sensor assemblies can be a time consuming and intricate surgical process.

FIGS. 1E-1G show various views of sensor assemblies 106, including electrodes 120, leads 122, and substrate 123. As shown in FIG. 1E, sensor assembly can include at least one electrode 120 and an electrical lead 122 that electrically couples the electrode to the control unit of the stroke detection system. FIG. 1F is a cutaway view of a sensor assembly showing three electrical leads. FIG. 1G shows a side cutaway view of the sensor assembly 106, including the electrode 120 and electrical lead 122. The sensor assembly includes a slim profile to facilitate placement under the patient's skin without causing pain or discomfort. Preferably the entire sensor assembly has a thickness less than 2 mm. In one embodiment, the sensor assembly has a thickness ranging from approximately 1 mm to 4 mm. The sensor assembly substrate is also preferably bendable or conformable to the shape of the patient's head, to further facilitate long-term, pain free implantation. For example, the substrate 123 can be manufactured with a variety of materials such as polymers or paper, including polyethylene, terephthalate, polyethelene naphthalate, polyimide, polyurethane, styrene-ethylene-butadiene-styrene, polydimethylsiloxane, polyetherimide, along with various fluoropolymers and copolymers. The leads 122 can comprise a variety of conductive materials, including metals, foils, conductive pastes, inks, or fluids, conductive polymers, or conductive nanomaterials and micro materials. The electrodes 120 also comprise conductive materials, which can include metal films, foils, or wires, or other conductive materials including gold, silver, silver chloride, platinum, platinum-iridium, rhodium, palladium, stainless steel, gold, tungsten, titanium, cobalt, or MP35N alloy. The electrodes can also be made with variants of conductive hydrogels or polymers such as polyanilines, polypyrroles, polythiophenes, or poly(3,4-ethylenedioxythiophene) polystyrene sulfonate (PEDOT:PSS).

The sensor assemblies can be encapsulated with a material to provide a protective mechanical barrier selected to minimize tissue inflammatory response. In some examples, the encapsulation material can comprise silicones and silicone-urethanes, ceramic casings, glass casings, and a variety of metal alloys such as titanium alloys. The surface of the electrode can be flat or micro structured with the aim of achieving better mechanical coupling and better impedance matching. Examples of microstructures include waves, pyramids, cephalopod (acetabulum-shaped) suckers, or insect inspired pulvilli structures. The structures can appropriately deform to provide optimal coupling. In some examples, the electrodes can include a flexible electrical insulator for the span of the conductor that is not in contact with the targeted region of the brain.

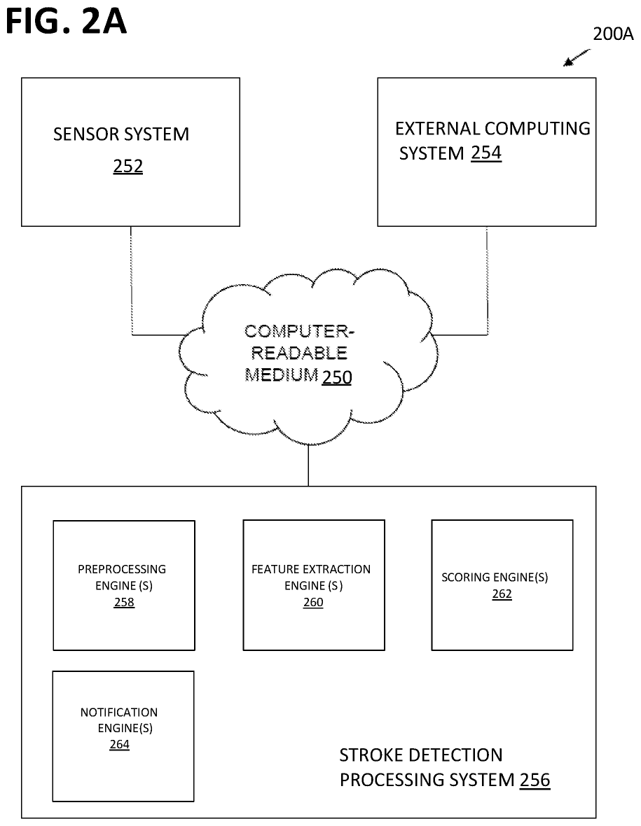

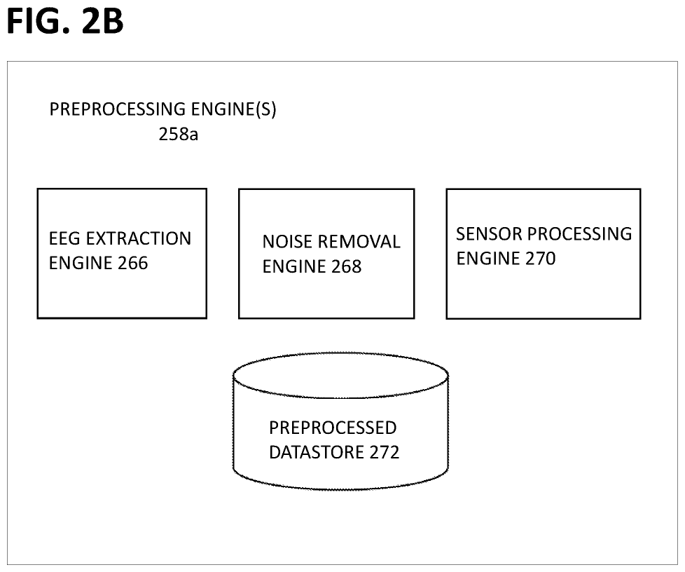

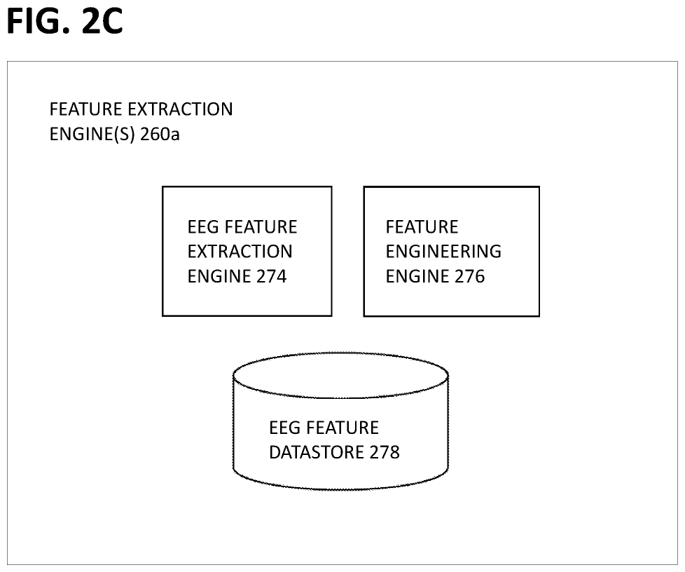

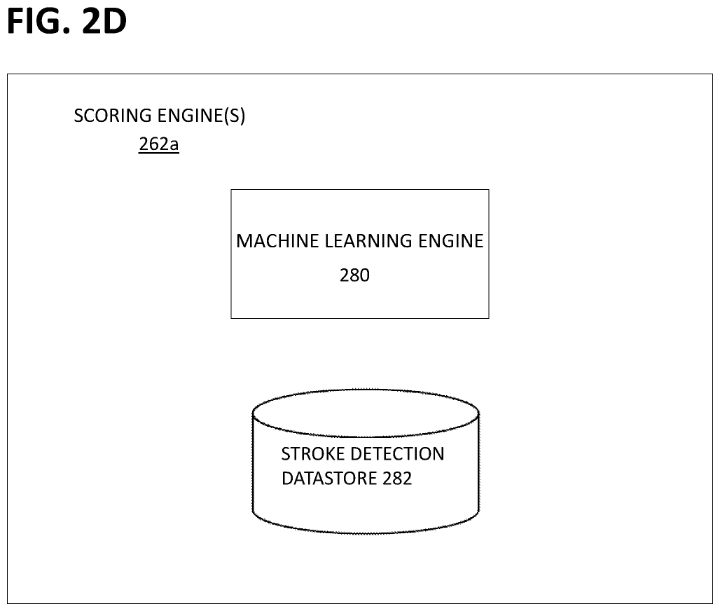

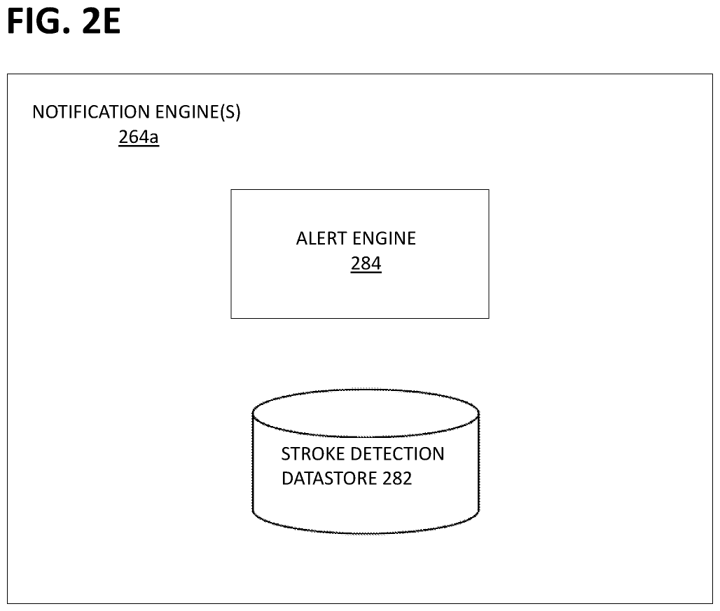

FIG. 2A is a diagram showing an example of a computing environment 200A configured to facilitate detecting the onset of stroke in a patient and providing feedback or a notification about the stroke event. The environment 200A includes a computer-readable medium 250, a sensor system 252, an external computing system 254, and a stroke detection processing system 256. One or more of the modules in the computing environment 200A may be coupled to one another or to modules not explicitly shown.

The computer-readable medium 250 and other computer readable media discussed herein are intended to represent a variety of potentially applicable technologies. For example, the computer-readable medium 250 can be used to form a network or part of a network. Where two components are co-located on a device, the computer-readable medium 250 can include a bus or other data conduit or plane. Where a first component is co-located on one device and a second component is located on a different device, the computer-readable medium 250 can include a wireless or wired back-end network or LAN. The computer-readable medium 250 can also encompass a relevant portion of a WAN or other network, if applicable.

The sensor system 252 may include one or more sensor assemblies or electrodes configured to measure electrical activity or biopotential voltage of a patient's brain. The sensor system 252 may include memory, one or more processors, and/or sensors to detect, measure, and/or record biopotential voltage of a patient's brain. The sensor system 252 may be implemented as an array of EEG electrodes implanted on or near a patient's brain. The sensor system 252 may be used as part of a stroke detection system configured to process and analyze EEG data to determine if the patient is having a stroke. In some embodiments, the sensor system includes four EEG electrodes, providing at least one electrode per lobe of the brain. In some embodiments, two sensor assemblies are positioned on the side of the head, and two sensor assemblies are positioned towards the top of the head. In some embodiments, bilateral sensor arrays, semicircular sensor arrays, and multi-sensor arrays are used. In some embodiments, arrays of 1, 2, 3, 4, 5, 6, 7, 8, 9, 10, 11-16, or 17-32 EEG electrodes are used.

The external computing system 254 may include a computer system configured to receive a stroke detection alert/notification and display or audibly alert a third party (e.g., a caregiver, loved one, or medical or emergency personnel) that the patient is having a stroke. The external computing system 254 may include memory, one or more processors, and a display device to display the stroke alert. The external computing system 254 may be implemented as part of a computer system, a smartphone, tablet, or wearable device. In some implementations, the external computing system 254 may receive the stroke detection alert and display or audibly announce to the third party that the patient is having a stroke. The external computing system 254 may also include information relevant to providing immediate care to the patient, including the patient's age, gender, physical description, precise geo-location, etc.

The stroke detection processing system 256 may include a computer system configured to process biopotential voltage, or other input and biomarkers, measured by the sensor system 252. As noted herein, the stroke detection processing system 256 may be configured to process and analyze EEG data. The stroke detection processing system 256 may include preprocessing engine(s) 258, feature extraction engine(s) 260, scoring engine(s) 262, and notification engine(s) 264. One or more of the modules of the stroke detection processing system 256 may be coupled to each other or to modules not shown.

As used herein, any “engine” may include one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specific or other considerations, an engine can be centralized or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used herein, “datastores” may include repositories having any applicable organization of data, including tables, comma-separated values (CSV) files, JSON files, XML files, traditional databases (e.g., SQL), or other applicable known or convenient organizational formats (e.g., European Data Format (edf, edf+). Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

The preprocessing engine(s) 258 may be configured to implement one or more automated agents configured to process measured biopotential voltage of the patient's brain from the sensor system 252 into EEG data. In some implementations, the preprocessing engine(s) 258 can be further configured to extract EKG data from the biopotential voltage or the EEG data. The preprocessing engine(s) 258 may be configured to remove noise and other undesirable characteristics from the biopotential voltage or EEG data. The preprocessing engine(s) 258 may be configured to infer noisy signals from specific noisy or corrupted electrodes by appropriately interpolating information from the surrounding neighboring electrodes with data interpolation methods (e.g., scalp surface Laplacian). The preprocessing engine(s) 258 can also be configured to use sensor data from the control unit to remove or ignore data that is inaccurate, corrupted, or unusable. For example, a pressure sensor, a proximity sensor, a temperature sensor on the sensor assembly or the control unit may provide an indication that pressure is being applied to the sensor assembly (e.g., the patient is lying down and applying pressure to the electrode) which may render the measurements inaccurate. The preprocessing engine(s) can use this information from the pressure sensor to remove or ignore the measured data when pressure was applied to the sensor assembly. The preprocessing engine(s) 258 may provide EEG data, EKG data, preprocessed data, and/or other data to other modules of the stroke detection processing system 256.

The feature extraction engine(s) 260 may implement one or more automated agents configured to extract EEG features from the EEG data. An “EEG feature,” as used herein, may include data points from the EEG data that correlate to frequencies, slopes, peaks and troughs. An “EEG feature” may be based on patient demographics. An “EEG feature” may be related to data connectivity metrics e.g., Granger causality, total EEG information flow. An “EEG feature” may be related to “PCA features,” e.g., those EEG features derived from a principal component analysis of EEG data. The feature extraction engine(s) 260 may be configured to compute a variety of metrics or features which can be aggregated to calculate an overall score, which in turn is trended over time and compared against predefined thresholds (such as, but not limited to, value thresholds and slope thresholds) or compared against the last known control baseline state as defined by an adaptive time window of data. For example, the feature extraction engine(s) 260 may be configured to compute metrics including, but not limited to, asymmetry, loss of high frequency power, increase of low frequency power, loss of amplitude, change in connectivity, and time. The feature extraction engine(s) 260 may provide EEG data, EKG data, computed metrics or features, and/or other data to other modules of the stroke detection processing system 256.

In some examples, the feature extraction engine(s) 260 may implement one or more automated agents configured to implement feature engineering to produce features for the stroke detection scoring model. There are many approaches to do feature engineering for building a scoring model, including hand designed features of Power Spectral Density (PSD) and ratios and spatial/temporal differences thereof, hand designed brain connectivity metrics and spatial/temporal differences thereof, hand designed features of Principle Component Analysis (PCA) and spatial/temporal differences thereof, automated feature exploration (e.g., using deep neural networks or other feature selection methods) and spatial/temporal differences thereof, and a hybrid feature engineering approach, namely combining hand designed features and automated feature exploration model architecture and spatial/temporal differences thereof.

The scoring engine(s) 262 may implement one or more automated agents configured to determine if the patient is having a stroke using extracted and processed EEG features. The scoring engine(s) 262 may acquire EEG features from the feature extraction engine(s) 260 and apply machine learning algorithms to determine if the patient is having a stroke. In some implementations, the scoring engine(s) 262 use a trained convolutional neural network and/or trained classifiers to classify a patient's EEG data into one or more identified categories of not having a stroke, likely having a stroke, having a stroke, etc. Examples of machine learning systems implemented by the scoring engine(s) 262 may include Decision Tree, Random Forest, Logistic Regression, Support Vector Machine, AdaBOOST, K-Nearest Neighbor (KNN), Quadratic Discriminant Analysis, Neural Network, etc., to determine if the patient is having a stroke. In some implementations, the scoring engine(s) 262 can determine the size of the stroke. For example, the scoring engine(s) 262 can evaluate how many of the sensor assemblies or electrodes are detecting stroke activity and the rate of core expansion and using information about the physical location of the sensor assemblies. This can determine if the stroke is a affecting a small or a large volume of brain tissue and can infer the affected arterial supply vessel, which may in turn aide in deciding for a treatment modality and level of care needed.

The notification engine(s) 264 may implement one or more automated agents configured to notify the patient or a third party to the patient that the patient is having a stroke. In some implementations, the notification engine(s) 264 may provide an electronic notification to an external computing device, such as a pc, smartphone, tablet, or emergency telephone system, that the patient is having a stroke. The notification engine(s) 264 may include relevant information in the notification, including the patient's age, gender, a physical description, accurate geo-location, etc. In another implementation, the notification engine(s) 264 may deliver a pre-recorded or predetermined audio message to an external computing device that the patient is having a stroke. In other embodiments, the notification engine(s) 264 can be configured to provide feedback on the device itself that the patient is having a stroke. For example, the notification engine(s) 264 can be configured to cause the stroke detection system to vibrate or emit an audible noise in the event of a stroke.

FIG. 2B is a diagram showing an example of the preprocessing engine(s) 258a. The preprocessing engine(s) 258a may include an EEG extraction engine 266, a noise removal engine 268, a sensor processing engine 270, and a preprocessed datastore 272. One or more of the modules of the preprocessing engine(s) 258a may be coupled to each other or to modules not shown.

The EEG engine 266 may implement one or more automated agents configured to extract EEG data from biopotential voltage of the patient's brain measured by the sensor assemblies. The EEG data extraction may be performed with a bipolar or a referential mode. When the referential mode is used a specific electrode or the average of values among or electrodes may be used as a reference value. The appropriate selection of the acquisition mode can assist with noise reduction and thus the system can switch to the less noisy mode in real time when needed. The digitized EEG data from the EEG engine 266 can be passed onto other engines in the preprocessing engine(s) 258a, including the noise removal engine 268 and sensor processing engine 270. Additionally, the EEG data can be stored in preprocessed datastore 272.

The noise removal engine 268 may implement one or more automated agents configured to remove noise or interferences from the EEG data. For example, noise components can be separated from the recorded EEG data and removed. In some examples, the noise can be filtered out of the EEG data, for example by using high-pass, low-pass, or notch filters. Noise can also be identified through a metric of overall statistical coherence of the data from all the recorded channels, where coherence lower than a specific threshold or higher than a specific threshold automatically implies that the signal is corrupted and/or noisy for a specific channel and has to be removed or inferred from the surrounding electrodes. The resulting clean EEG data can be passed onto other engines in the preprocessing engine(s) 258a, including the sensor processing engine 270. Additionally, the clean EEG data can be stored in preprocessed datastore 272.

The sensor processing engine 270 may implement one or more automated agents configured to apply sensor data to the EEG data or clean EEG data to remove or ignore signals that are inaccurate, unusable, or corrupted. For example, a pressure sensor on the sensor assembly or in the control unit may provide an indication that pressure is being applied to the sensor assembly (e.g., the patient is lying down and applying pressure to the electrode) which may render the measurements inaccurate. The sensor processing engine 270 can use this information from the pressure sensor to remove or ignore the measured data when pressure was applied to the sensor assembly. Additionally, the sensor modified EEG data can be stored in preprocessed datastore 272.

The preprocessed datastore 272 may be configured to store data related to stroke detection, including measured biopotential voltages, EEG data, clean EEG data, sensor data, and sensor modified EEG data.

FIG. 2C is a diagram showing an example of a feature extraction engine(s) 260a. The feature extraction engine(s) 206a may include EEG feature extraction engine 274, feature engineering engine 276, and an EEG feature datastore 278. One or more of the modules of the feature extraction engine(s) 260a may be coupled to each other or to modules not shown.

The EEG feature extraction engine 274 may implement one or more automated agents configured to determine or extract EEG features from the EEG data. The EEG features may comprise, for example, metrics including asymmetry, loss of high frequency power, increase of low frequency power, loss of amplitude, change in connectivity, and time. The EEG feature extraction engine 274 can communicate these features or metrics to the EEG feature datastore 278.

The feature engineering engine 276 may implement one or more automated agents configured to implement feature engineering to produce features for the scoring model. There are many approaches to do feature engineering for building a scoring model, including hand designed features of Power Spectral Density (PSD) and ratios and spatial/temporal differences thereof, hand designed brain connectivity metrics and spatial/temporal differences thereof, hand designed features of Principle Component Analysis (PCA) and spatial/temporal differences thereof, automated feature exploration (e.g. using deep neural networks or other feature selection methods) and spatial/temporal differences thereof, and a hybrid feature engineering approach, namely combining hand designed features and automated feature exploration model architecture and spatial/temporal differences thereof. The feature engineering engine 276 can communicate these engineered features to the EEG feature datastore 278.

The EEG feature datastore 278 may be configured to store data related to EEG features or metrics, and engineered features from the modules described above.

FIG. 2D is a diagram showing an example of the scoring engine(s) 262a. The scoring engine(s) 262a may acquire EEG features or metrics, and engineered features data from the EEG feature extraction engine 274 and the feature engineering engine(s) 276 described above. The scoring engine(s) 262a may include a machine learning engine 280 and a stroke detection datastore 282.

The machine learning engine 280 may implement one or more automated agents configured to use machine learning techniques to classify whether or not a patient is having a stroke. In some implementations, the machine learning engine 280 may acquire EEG features, EEG metrics, and engineered features data from the EEG feature extraction engine 274 and the feature extraction engine(s) 276. Using a trained classifier, the machine learning engine 280 may provide an identifier (e.g., a statistical or other score) that associates the patient's electrical brain activity with a specified category of stroke (e.g., no stroke or stroke). As examples, the machine learning engine 280 may use a classifier trained to correlate various EEG features with whether the patient is having a stroke. The machine learning engine 280 may incorporate one or more machine learning techniques. Examples of such techniques include Convolutional Neural Networks (CNN), Decision Tree, Random Forest, Logistic Regression, Support Vector Machine, AdaBOOST, K-Nearest Neighbor (KNN), Quadratic Discriminant Analysis, Neural Network, etc. The machine learning engine 280 can provide an output with stroke score. The output can be, for example, a binary score or a linear score.

The machine learning engine 280 may use a specified amount of EEG data and EEG features to determine if the patient is having a stroke. While the sensor assemblies of the stroke detection system are continuously monitoring EEG activity from the patient's brain, the machine learning engine 280 may analyze only a rolling window of the EEG data and EEG features. For example, the machine learning engine 280 may receive a rolling window of EEG data and EEG features, for example, about 1, 2, 3, 4, 5, 6, 7, 8, 9, 10-20, 20-30, 30-40, 40-50, or 50-60 seconds of data and features and use only the data from that rolling window to make a determination. In another embodiment, the rolling window of data can comprise, for example, about 1, 2, 3, 4, 5, 6, or 7-15 minutes of data. The amount of data analyzed by the machine learning algorithm can be used to increase accuracy, at the expense of potentially delaying life-saving medical care to the patient. For example, analyzing only 1 second of data may result in a false positive for stroke, but would also shorten the time for sending out an alert if the patient is having a stroke. In another example, multiple continuous rolling windows of EEG data can be evaluated before a determination of “stroke” or “no stroke” can be made. A stroke determination can be made when a predetermined number of consecutive rolling time windows all return a positive “stroke” score from the classifier. In one example, the stroke detection system may return a determination of “stroke” when 5-10 continuous rolling time windows of EEG data return a positive or “stroke” classification from the scoring model.

In some implementations, the machine learning engine may initiate a confirmation test if a stroke is detected. A confirmation test may take a second look at new EEG data and engineered features coming from the patient by passing the most recent EEG data and engineered features into the classifier to confirm that a stroke is occurring. In some embodiments, parameters of the data can be changed, such as the length of the rolling window of data that is evaluated, the sampling frequency, etc. If the anomaly is confirmed as persistent (e.g., the confirmation test confirms that a stroke is occurring), then the stroke score can be stored in the stroke detection datastore 282, and the notification engine(s) 264 can be activated.

The stroke detection datastore 282 may be configured to store data relating to the stroke score from the machine learning engine 280 (e.g., the output from the machine learning engine). In some implementations, the stroke score is a binary labeling score (e.g., a score of 1 for when the patient is having a stroke and a score of 0 for when the patient is not having a stroke). In other implementations, the stroke score is a linear labeling score (e.g., a score ranging from 0 to 10, where 0 indicates that the patient is unlikely to be having a stroke and a score of 10 indicates that the patient is likely having a stroke). In another embodiment, the stroke score is a multilabel labeling score (e.g., “stroke”, “no stroke”, “non-stroke/seizure”, “non-stroke/walking”, “non-stroke/sleep”, “non-stroke/chewing”, “non-stroke/blinking”, “non-stroke/facial muscle activity, etc.).

FIG. 2E is a diagram showing an example of the notification engine(s) 264a. The notification engine(s) 264a may include an alert engine 284 and an alert datastore 286. One or more of the modules of the notification engine(s) 264a may be coupled to each other or to modules not shown.

The alert engine 284 may implement one or more automated agents configured to send, transmit, and deliver a stroke alert, notification, or feedback to a patient or a third party in the event that the machine learning engine above determines that the patient is having a stroke. In one example, the alert engine provides a digital message, such as a sms/mms alert or an email alert to the patient or a third party, including loved ones, caregivers, or emergency/medical personnel. The digital message can include the notification that the patient is having a stroke, and can further included relevant details about the patient including a physical description, age, gender, geo-location, and contact information. In other example, the alert can comprise a pre-recorded or predetermined voice message or audible alert with the relevant information described above. The alert engine is configured to provide all the necessary information to the patient or another to ensure that the patient can be located and receive appropriate medical assistance in a timely manner. In another example, the alert engine 284 provides feedback and can be configured to cause the stroke detection system to vibrate, to emit a repeated flash of light or emit an audible noise in the event of a stroke.

The primary goal of the notification system is to bring the patient as fast as possible to the closest, most appropriate treatment center. In some examples, the third party to be alerted, notified, or contacted by the stroke detection system and the alert engine are the hospital closest to the patient, personal emergency contacts, or direct notification of PSAP or EMS via a data link or text message. In another example, the stroke detection system activates a local patient alert system. In another example, the stroke detection system communicates the alert to a service center, who in turn can augment the alert with patient specific background data (e.g., age, gender, medical history, current medication, etc.), activate the appropriate resources and notify the appropriate treatment center.

The stroke detection datastore 282 may be configured to store data related to stroke scores from the machine learning engine, and also may be configured to store relevant information about the patient, including a physical description, age, gender, geo location, and contact information.

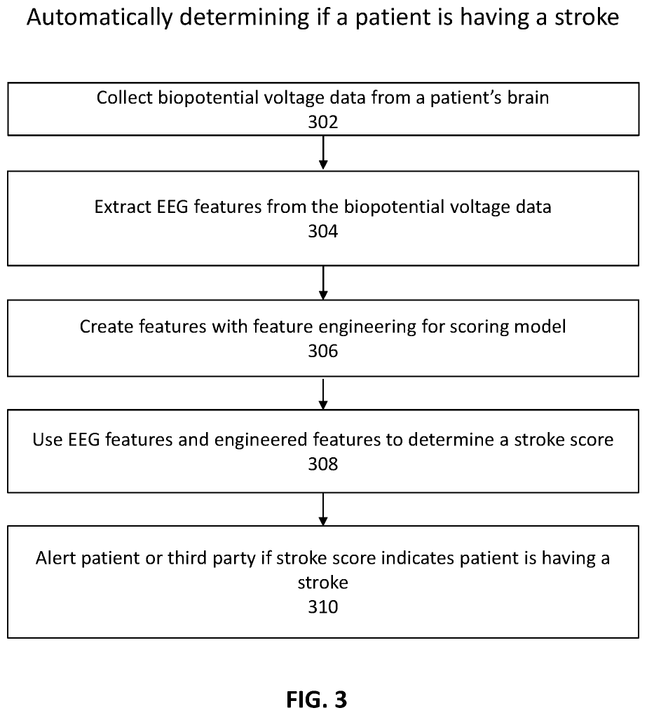

FIG. 3 illustrates one example of a method for automatically determining if a patient is having a stroke. This method may be automatically implemented by a system, such the stroke detection system 100 of FIG. 1A or one or more of the systems in the computing environment 200A, shown in FIG. 2A. At an operation 302, the system may automatically collect biopotential voltage data from a patient's brain. The biopotential voltage data may be collected directly from the patient (e.g., using sensors or electrodes) or indirectly (e.g., by receiving biopotential voltage data of the patient taken by another, etc.).

The biopotential voltage data may be prepared for further processing. For example, noise, interferences, or other unusable or corrupted data may be removed. At an operation 304, EEG features may be extracted from the biopotential voltage data using an EEG extraction engine. The EEG features or metrics may include, but not be limited to asymmetry, loss of high frequency power, increase of low frequency power, loss of amplitude, change in connectivity, and time.

At an operation 306, additional features for the scoring model may be created with feature engineering. In one example, feature engineering can include hand designed features of Power Spectral Density (PSD) and ratios and spatial/temporal differences thereof, and hand designed brain connectivity metrics and spatial/temporal differences thereof, and hand designed features of principal component analysis (PCA) can be implemented to obtain the engineered features that will be used by the scoring model. In one example, the feature engineering engine may implement automated feature exploration (e.g., using deep neural networks or other feature selection methods) to produce the features for the scoring model. Alternatively or in combination, a third approach may include using both PCA and automated feature exploration (e.g., a hybrid approach) to produce the features that will be ultimately passed on to the scoring model.

At an operation 308 the EEG features and the engineered features may be used exclusively or in combination with any other feature or metric described herein. The features may be provided to the scoring engine to determine a stroke score that indicates how likely it is that a patient is having a stroke. In some examples, the stroke detection system can run a confirmation test to confirm that a stroke is occurring. The stroke confirmation test can comprise continuously delivering new EEG data and engineered features to the scoring engine to confirm the presence of a stroke condition in the patient. If a stroke is not confirmed by the confirmation test, then the initial stroke positive can be flagged as an anomaly or as transient ischemia for later review.

At an operation 310, the stroke score may then be output. In some variations this output comprises an alert that provides a digital message or recorded/audible message, such as a sms/mms alert, an email alert, or a voice message to the patient or a third party, including loved ones, caregivers, or emergency/medical personnel. The alert can include relevant details about the patient including a physical description, age, gender, geo location, and contact information.

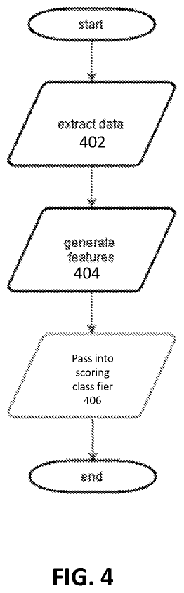

FIG. 4 is another flowchart that shows the overall scoring system described above and describes a method of extracting and generating data to be passed into a stroke scoring system.

At an operation 402 of FIG. 4, data can be extracted for use by the scoring system. The data can include EEG data and metrics. Next, at step 404 of FIG. 4, the detection system can generate features from the extracted data, including engineered features, as described above. Finally, at step 406 of FIG. 4, the features can be passed into the scoring classifier to determine a stroke score.

Additionally, the techniques described here may be implemented in hardware or software, or a combination of the two. The techniques may be implemented in computer programs executing on programmable computers that each includes a processor, a storage medium readable by the processor (including volatile and nonvolatile memory and/or storage elements), and suitable input and output devices. Program code is applied to data entered using an input device to perform the functions described and to generate output information. The output information is applied to one or more output devices.

Each program can be implemented in a high level procedural or object-oriented programming language to operate in conjunction with a computer system. However, the programs can be implemented in assembly or machine language, if desired. In any case, the language may be a compiled or interpreted language.

Each such computer program can be stored on a storage medium or device (e.g., CD-ROM, hard disk or magnetic diskette) that is readable by a general or special purpose programmable computer for configuring and operating the computer when the storage medium or device is read by the computer to perform the procedures described. The system also may be implemented as a computer-readable storage medium, configured with a computer program, where the storage medium so configured causes a computer to operate in a specific and predefined manner.

Thus, any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the invention and that methods and structures within the scope of these claims and their equivalents be covered thereby.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and/or methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the patent matter may be practiced. As mentioned, other embodiments may be utilized and derived there from, such that structural and logical substitutions and changes may be made without departing from the scope of this disclosure. Such embodiments of the inventive patent matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

A complete listing of the claims follows:

1. A method of automatically determining if a subject is having a stroke, the method comprising the steps of:
acquiring, in a computing device, biopotential voltage data from the subject's brain;
determining, in the computing device, EEG features or metrics from the biopotential voltage data;
creating, in the computing device, engineered features from the EEG features or metrics;
applying the engineered features to a classifier of the computing device;
outputting from the computing device a stroke score for the subject; and
in the event that the stroke score indicates that the subject is having a stroke, providing feedback that the subject is having a stroke.

2. The method of claim 1, wherein the feedback comprises sending a notification to the subject or a third party that the subject is having a stroke

3. The method of claim 1, wherein outputting comprises outputting a binary score indicating that the subject is likely having a stroke or likely not having a stroke.

4. The method of claim 1, wherein outputting comprises outputting a linear score indicating along a scale how likely it is that the subject is having a stroke.

5. The method of claim 1, wherein the creating engineered features step further comprises taking a principal component analysis (PCA) of the EEG features or metrics.

6. The method of claim 1, wherein creating engineered features comprises power spectral density (PSD) ratios and channel connectivity metrics and spatial/temporal differences thereof.

7. The method of claim 1, wherein the biopotential voltage is acquired from at least one electrode implanted under the scalp of the subject.

8. The method of claim 1, wherein applying the engineered features to the classifier comprises applying either a binary classifier or a linear classifier to the engineered features.

9. The method of claim 2, wherein the notification comprises a digital text message to an external device that the subject is having a stroke.

10. The method of claim 9, wherein the notification includes relevant information about the subject selected from the group consisting of age, gender, physical description of the subject, and physical location of the subject.

11. The method of claim 2, wherein the notification comprises a voice or audible message to an external device that the subject is having a stroke.

12. The method of claim 11, wherein the notification includes relevant information about the subject selected from the group consisting of age, gender, physical description of the subject, and physical location of the subject.

13. A non-transitory computing device readable medium having instructions stored thereon for determining if a subject is having a stroke, wherein the instructions are executable by a processor to cause a computing device to:
acquire biopotential voltage data from the subject's brain;
determine EEG features or metrics from the biopotential voltage data;
create engineered features from the EEG features or metrics;
apply the engineered features to a classifier of the computing device;
output a stroke score for the subject; and
in the event that the stroke score indicates that the subject is having a stroke, provide feedback that the subject is having a stroke.

14-23. (canceled)

24. A device configured to detect the onset of stroke, comprising:
at least one electrode configured to be implanted under a subject's scalp to measure biopotential voltage of the subject's brain;
a control unit electrically coupled to the at least one electrode, the control unit comprising a processor, a power source, communications hardware configured to transmit and receive data to and from an external device, and at least one memory storage device disposed in a housing, wherein the control unit is configured to:
acquire, in the at least one memory storage device, biopotential voltage data from the at least one electrode;
determine, with the processor, EEG features or metrics from the biopotential voltage data;
create, with the processor, engineered features from the EEG features or metrics;
apply, with the processor, the engineered features to a classifier stored on at least one memory storage device;
output, with the processor, a stroke score for the subject; and
in the event that the stroke score indicates that the subject is having a stroke, provide, with the communications hardware, feedback that the subject is having a stroke.

25. The device of claim 24, wherein the feedback comprises a notification to the subject or a third party that the subject is having a stroke

26-31. (canceled)

32. The device of claim 24, further comprising at least one sensor.

33. The device of claim 32, wherein the sensor is an accelerometer, a pressure sensor, a microphone, or a GPS sensor.

34. The device of claim 24, wherein the at least one electrode comprises at least 2 electrodes configured for placement on each temporal lobe on the subject's brain.

35. The device of claim 24, wherein the at least one electrode comprises at least 2 electrodes configured for placement on each frontal parietal lobe of the subject's brain.

36. The device of claim 24, wherein the at least one electrode has a thickness between about 10 microns and about 2 mm.

37-38. (canceled)
